(19) United States
(12) Patent Application Publication
(10) Pub. No.: US 2022/0218530 A1
(43) Pub. Date: Jul. 14, 2022

(54) Perforated Collagen Wound Interface For Use With Negative Pressure Wound Therapy

(71) Applicant: KCI Licensing, Inc., San Antonio, TX (US)

(72) Inventor: Christopher Brian LOCKE, Bournemouth (GB)

(21) Appl. No.: 17/607,795

(22) PCT Filed: May 21, 2020
(86) PCT No.: PCT/US2020/033982

§ 371 Date: Oct. 29, 2021

Related U.S. Application Data
(60) Provisional application No. 62/852,529, filed on May 24, 2019.

(51) Int. Cl. A61F 13/02; A61M 1/00; A61F 13/00; A61L 15/32; A61L 15/44

(52) U.S. Cl. CPC A61F 13/0216; A61F 13/0289; A61M 1/915; A61L 2300/104; A61L 15/325; A61L 15/44; A61F 13/00063

(57) ABSTRACT

The present disclosure relates generally to wound dressings that reduce the pressure drop observed during use in negative pressure wound therapy (NPWT). The wound dressings include a first layer and a second layer; wherein each of the first layer and the second layer independently comprise a biopolymer and a plurality of perforations, wherein each perforation comprises a width and an external perimeter, and wherein the first layer and the second layer are adjoined such that the external perimeter of each perforation in the first layer does not overlap or intersect with the external perimeter of each perforation in the second layer.

### CROSS-REFERENCE TO RELATED APPLICATIONS

This application claims the benefit of and priority to U.S. Patent Application No. 62/852,529, filed on May 24, 2019, the contents of which are incorporated herein by reference in their entirety.

### TECHNICAL FIELD

The present technology relates generally to wound dressings that reduce the pressure drop observed during use in negative pressure wound therapy (NPWT), and methods of using the same. Kits for use in practicing the methods are also provided.

### BACKGROUND

The following description of the background of the present technology is provided simply as an aid in understanding the present technology and is not admitted to describe or constitute prior art to the present technology.

A wide variety of materials and devices, generally characterized as “dressings,” are generally known in the art for use in treating a wound or other disruption of tissue. Such wounds may be the result of trauma, surgery, or disease, and may affect skin or other tissues. In general, dressings may control bleeding, absorb wound exudate, ease pain, assist in debriding the wound, protect wound tissue from infection, or otherwise promote healing and protect the wound from further damage. Negative pressure wound therapy (NPWT) is a type of wound therapy that involves applying a negative pressure to a wound site to promote wound healing. Clinical studies have shown that providing a reduced pressure in proximity to a wound site can assist in wound healing by promoting blood flow to the wound, stimulating the formation of granulation tissue, and encouraging the migration of healthy tissue over the wound.

### SUMMARY OF THE PRESENT TECHNOLOGY

In one aspect, the present disclosure provides a wound dressing that includes a first layer 130 and a second layer 140, wherein each of the first layer 130 and the second layer 140 independently include a biopolymer 100 and a plurality of perforations 110, each perforation 110 includes a width and an external perimeter, and the first layer 130 and the second layer 140 are adjoined such that the external perimeter of each perforation 110 in the first layer 130 does not overlap or intersect with the external perimeter of each perforation 110 in the second layer 140.

Additionally or alternatively, in some embodiments, each of the first layer 130 and the second layer 140 independently comprise a wound-facing side and an environmental-facing side. Additionally or alternatively, in some embodiments, the wound-facing side of the first layer 130 is coupled with the environmental-facing side of the second layer 140.

Additionally or alternatively, in some embodiments, the width of each perforation 110 in each of the first layer 130 and the second layer 140 is about 2 mm to about 5 mm. Additionally or alternatively, in some embodiments, the width of each perforation 110 in each of the first layer 130 and the second layer 140 may independently be about 2 mm, about 2.5 mm, about 3 mm, about 3.5 mm, about 4 mm, about 4.5 mm, about 5 mm, or any range including and/or in between any two of these values.

Additionally or alternatively, in some embodiments, the perforations 110 in each of the first layer 130 and the second layer 140 has a pitch of about 5 mm to about 10 mm. Additionally or alternatively, in some embodiments, the perforations 110 in each of the first layer 130 and the second layer 140 has a pitch of about 5 mm, about 5.5 mm, about 6 mm, about 6.5 mm, about 7 mm, about 7.5 mm, about 8 mm, about 8.5 mm, about 9 mm, about 9.5 mm, about 10 mm, or any range including and/or in between any two of these values. Additionally or alternatively, in some embodiments, the perforations 110 in each of the first layer 130 and the second layer 140 has a pitch of about 10 mm.

Additionally or alternatively, in some embodiments, the shape of the perforations 110 in each of the first layer 130 and the second layer 140 is independently a circle, a triangle, a quadrilateral, or a polygon (e.g., a pentagon, a hexagon, a heptagon, an octagon, a nonagon, or a decagon).

Additionally or alternatively, in some embodiments, the first layer 130 further includes channels 160, wherein each channel is configured to intersect with at least one perforation along the environmental-facing side of the first layer 130.

Additionally or alternatively, in some embodiments, the biopolymer 100 of each of the first layer 130 and second layer 140 are independently one or more of a collagen, an oxidized cellulose, a polysaccharide, chitosan, gelatin, hyaluronic acid, or any combination thereof.

Additionally or alternatively, in some embodiments, each of the first layer 130 and the second layer 140 independently may include about 0.1 wt. % to about 100 wt. % biopolymer 100. Additionally or alternatively, in some embodiments, the amount of biopolymer 100 in each of the first layer 130 and the second layer 140 may be about 0.1 wt. %, about 0.5 wt. %, about 1 wt. %, about 2.5 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 98 wt. %, about 99 wt. %, about 100 wt. %, or any range including and/or in between any two of the preceding values.

Additionally or alternatively, in some embodiments, each of the first layer 130 and the second layer 140 independently has a thickness of about 1 mm to about 3 mm. Additionally or alternatively, in some embodiments, each of the first layer 130 and the second layer 140 independently has a thickness of about 1 mm, about 1.5 mm, about 2 mm, about 2.5 mm, about 3 mm, or any range including and/or in between any two of the preceding values. Additionally or alternatively, in some embodiments, the first layer has a different thickness than the second layer.

Additionally or alternatively, in some embodiments, one or more of the first layer 130 and the second layer 140 comprises a silver compound. Additionally or alternatively, in some embodiments, one or more of the first layer 130 and the second layer 140 may include about 0.1 wt. % to about 3 wt. % of the silver compound. Additionally or alternatively, in some embodiments, one or more of the first layer 130 and the second layer 140 may include about 0.1 wt. %, about 0.25 wt. %, about 0.50 wt. %, about 0.75 wt. %, about 0.8 wt. %, about 0.85 wt. %, about 0.9 wt. %, about 0.95 wt. %, about 1 wt. %, about 1.25 wt. %, about 1.5 wt. %, about 1.75 wt. %, about 2 wt. %, about 2.25 wt. %, about 2.5 wt. %, about 2.75 wt. %, about 3 wt. %, or any range including and/or in between any two of the preceding values, of the silver compound.

Additionally or alternatively, in some embodiments, the silver compound may include one or more pharmaceutically acceptable silver salts. Additionally or alternatively, in some embodiments, the one or more pharmaceutically acceptable silver salts is selected from the group consisting of silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver p-aminobenzoate, silver p-aminosalicylate, nanocrystalline silver, any pharmaceutically acceptable salt thereof, and any combination thereof.

Additionally or alternatively, in some embodiments, the first layer 130 and the second layer 140 are adjoined with a solution 150. Additionally or alternatively, in some embodiments, the solution 150 contains an adhesive. Additionally or alternatively, in some embodiments, the solution 150 is water, ethanol, acetic acid, a sugar, a liquid alginate solution, or any combination thereof. Additionally or alternatively, in some embodiments, the solution 150 is applied to the wound dressing via spray application or screen printing. Additionally or alternatively, in some embodiments, the wound dressing is freeze-dried or re-freeze dried after the solution 150 is applied to adjoin the first layer 130 and the second layer 140.

Additionally or alternatively, in some embodiments, the solution 150 further includes at least one additive. Additionally or alternatively, in some embodiments, the at least one additive is an antimicrobial agent, an antioxidant, a signaling protein, or any combination thereof.

Additionally or alternatively, in some embodiments, the solution 150 further comprises an antimicrobial agent. Additionally or alternatively, in some embodiments, the solution 150 comprises about 0.01% w/v to about 9% w/v of the antimicrobial agent. Additionally or alternatively, in some embodiments of the wound dressing disclosed herein, the antimicrobial agent may comprise about 0.01% w/v, about 0.1% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, or any range including and/or in between any two of these values. Additionally or alternatively, in some embodiments, the antimicrobial agent includes one or more of tetracycline, penicillins, terramycins, erythromycin, bacitracin, neomycin, polymycin B, mupirocin, clindamycin, colloidal silver, silver sulfadiazine, chlorhexidine, povidone iodine, triclosan, sucralfate, quaternary ammonium salts, pharmaceutically acceptable silver salts, or any combination thereof.

Additionally or alternatively, in some embodiments, the solution 150 further comprises an antioxidant. Additionally or alternatively, in some embodiments, the solution 150 comprises up to about 9% w/v of the antioxidant. Additionally or alternatively, in some embodiments, the solution 150 comprises about 1% w/v to about 9% w/v of the antioxidant. Additionally or alternatively, in some embodiments of the wound dressing disclosed herein, the antioxidant may comprise about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, or any range including and/or in between any two of these values. Additionally or alternatively, in some embodiments, the antioxidant includes one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or any combination thereof.

Additionally or alternatively, in some embodiments, the anthocyanins are selected from the group consisting of cyanidin, delphinidin, malvidin, pelargonidin, peonidin, petunidin, and any combination thereof.

Additionally or alternatively, in some embodiments, the flavanols are selected from the group consisting of catechin, epicatechin, theaflavin, thearubigins, gallocatechin, epigallocatechin, or any gallate ester thereof, and any combination thereof.

Additionally or alternatively, in some embodiments, the flavanones are selected from the group consisting of eriodictyol, hesperetin, naringenin, and any combination thereof.

Additionally or alternatively, in some embodiments, the flavones are selected from the group consisting of apigenin, luteolin, tangeritin, and any combination thereof.

Additionally or alternatively, in some embodiments, the flavonols are selected from the group consisting of isorhamnetin, kaempferol, myricetin, proanthocyanidins, quercetin, rutin, and any combination thereof.

Additionally or alternatively, in some embodiments, the isoflavone phytoestrogens are selected from the group consisting of daidzein, genistein, glycitein, and any combination thereof.

Additionally or alternatively, in some embodiments, the phenolic acids are selected from the group consisting of chicoric acid, chlorogenic acid, cinnamic acid, ellagic acid, ellagitannins, gallic acid, gallotannins, rosmarinic acid, salicylic acid, or any ester thereof, and any combination thereof.

Additionally or alternatively, in some embodiments, the stilbenoids are selected from the group consisting of resveratrol, pterostilbene, and any combination thereof.

Additionally or alternatively, in some embodiments, the solution 150 further includes a signaling protein. Additionally or alternatively, in some embodiments, the solution 150 comprises about 1% w/v to about 9% w/v of the signaling protein. Additionally or alternatively, in some embodiments of the wound dressing disclosed herein, the signaling protein may comprise about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, or any range including and/or in between any two of these values. Additionally or alternatively, in some embodiments, the signaling protein includes one or more of platelet-derived growth factor (PDGF), transforming growth factor beta (TGFβ), fibroblast growth factors (FGFs), epidermal growth factor (EGF), or any combination thereof.

Additionally or alternatively, in some embodiments, the fibroblast growth factors (FGFs) comprise one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGFS), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or any combination thereof.

Additionally or alternatively, in some embodiments, the wound dressing is configured for use in negative pressure wound therapy.

Additionally or alternatively, in some embodiments, application of the wound dressing causes about 50% to about 100% reduction in a pressure drop observed in negative pressure wound therapy compared to that observed with (a) a control wound dressing that does not comprise a plurality of perforations 110, or (b) a control wound dressing comprising a first layer 130 and a second layer 140, wherein each of the first layer 130 and the second layer 140 independently comprise a plurality of perforations 110, and wherein at least one perforation in the first layer 130 overlaps or intersects with a perforation in the second layer 140. Additionally or alternatively, in some embodiments of the wound dressing disclosed herein, the application of the wound dressing of the present technology causes about 50%, about 52%, about 54%, about 56%, about 58%, about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, about 100%, or any range including and/or in between any two of these values, reduction in pressure drop observed in negative pressure wound therapy compared to that observed with a control wound dressing.

In another aspect, the present disclosure provides a wound dressing that includes a first layer 130 and a second layer 140, wherein each of the first layer 130 and the second layer 140 independently include a biopolymer 100 and a plurality of perforations 110, each perforation 110 includes a width and an external edge, and the first layer 130 and the second layer 140 are mated such that the external edge of each perforation 110 in the first layer 130 does not overlap or intersect with the external edge of each perforation 110 in the second layer 140.

In another aspect, the present disclosure provides an apparatus for treating a wound, wherein the apparatus includes a first layer 130 and a second layer 140, wherein each of the first layer 130 and the second layer 140 independently include a biopolymer 100 and a plurality of perforations 110, each perforation 110 includes a width and an external edge, and the first layer 130 and the second layer 140 are mated such that the external edge of each perforation 110 in the first layer 130 does not overlap or intersect with the external edge of each perforation 110 in the second layer 140.

In another aspect, the present disclosure provides a method for treating a wound in a subject in need thereof, comprising administering to the wound a wound dressing of any embodiment disclosed herein. Additionally or alternatively, in some embodiments, the wound is an acute wound or a chronic wound. Additionally or alternatively, in some embodiments, the wound dressing is applied directly to the wound.

In another aspect, the present disclosure provides a method for treating a wound in a subject in need thereof, comprising administering to the wound an apparatus of any embodiment disclosed herein. Additionally or alternatively, in some embodiments, the wound is an acute wound or a chronic wound. Additionally or alternatively, in some embodiments, the apparatus is applied directly to the wound.

In another aspect, the present disclosure provides a method for making a wound dressing comprising, providing a first layer 130 and a second layer 140, generating a plurality of perforations 110 in each of the first layer 130 and the second layer 140, wherein each perforation comprises a width and an external perimeter, and combining the first layer 130 and the second layer 140 such that the external perimeter of each perforation in the first layer 130 does not overlap or intersect with the external perimeter of each perforation in the second layer 140.

Also provided herein are kits comprising the wound dressings of any embodiments disclosed herein and instructions for use.

### BRIEF DESCRIPTION OF THE DRAWINGS

FIG. 1A shows a diagrammatic representation of a cross-sectional view of an embodiment of a wound dressing of the present technology as well as the current state of the art.

FIG. 1B shows a diagrammatic representation of a cross-sectional view of an embodiment of a wound dressing of the present technology.

FIG. 1C shows a diagrammatic representation of an embodiment of a wound dressing of the present technology.

FIG. 2A shows the test set up for a standard PROMOGRAN material (control ORC/collagen, non-perforated). Pressure (mm Hg) was measured continuously for standard PROMOGRAN® under NPWT with instilled saline at 2.5 mL/hour for 24 hours.

FIG. 2B demonstrates the pressure drop observed at various locations across the control dressing under negative pressure.

FIG. 2C shows the test set up for the wound dressing of the present technology. Foam was used under the test item to allow negative pressure to manifold prior to measurement. NPWT unit is set to 125 mm Hg with no saline instilled, and pressure recorded is 126 mm Hg.

FIG. 2D demonstrates that as the pressure (125 mm Hg) was applied to the wound dressing of the present technology, the pressure stabilized to 126 mm Hg.

FIG. 2E demonstrates that the pressure applied to the wound dressing of the present technology stabilized to 126.3 mm Hg after the delivery of 10 mL saline. The perforated regions of the wound dressing became translucent and improved the manifolding over the dressing.

FIG. 2F demonstrates that the pressure applied to the wound dressing of the present technology stabilized to 126 mm Hg after the delivery of an additional 5 mL saline after a 10 minute swell period.

FIG. 2G demonstrates that the pressure applied to the wound dressing of the present technology stabilizes to 124.8 mm Hg after the delivery of 30 mL saline. The pressure was monitored for 3 hours after the addition of saline with no detected reduction in pressure.

### DETAILED DESCRIPTION

It is to be appreciated that certain aspects, modes, embodiments, variations and features of the present methods are described below in various levels of detail in order to provide a substantial understanding of the present technology.

“Negative pressure” may refer to a pressure less than a local ambient pressure, such as the ambient pressure in a local environment that is external to a sealed therapeutic environment provided by a dressing. Additionally or alternatively, in some embodiments, the local ambient pressure may also be the atmospheric pressure proximate to a wound site. Additionally or alternatively, in some embodiments, the local ambient pressure may also be less than a hydrostatic pressure associated with a wound site. Additionally or alternatively, in some embodiments, NPWT may provide a number of benefits, including, but not limited to, migration of epithelial and subcutaneous tissues, improved blood flow, and micro-deformation of tissue at a wound site. These benefits may increase development of granulation tissue and reduce healing times. Additionally or alternatively, in some embodiments, a negative pressure applied across a wound, via the NPWT device may be effective to induce macrostrain and microstrain at wound site, as well as remove exudates and other fluids from the wound site. Examples of setups for use with NPWT are disclosed in U.S. Pat. Nos. 7,534,240, 8,188,331, 8,529,526, and EP Patent 1758638, each incorporated by reference herein in their entirety.

The present disclosure is directed to multi-layer wound dressings (e.g., 2 layers, 3 layers, etc.), wherein each layer includes a plurality of perforations 110, which are adjoined such that each perforation in the first layer 130 does not overlap or intersect with each perforation in the second layer 140. The wound dressings of the present technology advantageously exhibit improved manifolding and decreased pressure drop observed in NPWT. The wound dressings of the present technology are able to impart constant pressure distribution across a wound site upon application. Further, the wound dressings of the present technology exhibit increased contact across the surface of a wound. The multi-layered construction of the wound dressings may further include at least one additive in an adhesive solution 150 to provide wound modulating components.

In one aspect, the present disclosure is directed to wound dressings that include a first layer 130 and a second layer 140, wherein each of the first layer 130 and the second layer 140 independently include a biopolymer 100 and a plurality of perforations 110, each perforation includes a width and an external perimeter, and the first layer 130 and the second layer 140 are adjoined such that the external perimeter of each perforation in the first layer 130 does not overlap or intersect with the external perimeter of each perforation in the second layer 140.

In one aspect, the present disclosure is directed to wound dressings that include a first layer 130 and a second layer 140, wherein each of the first layer 130 and the second layer 140 independently include a biopolymer 100 and a plurality of perforations 110, each perforation includes a width and an external perimeter, and the first layer 130 and the second layer 140 are adjoined such that the external perimeter of each perforation in the each of the first layer 130 and the second layer 140 overlap or intersect up to about 50%.

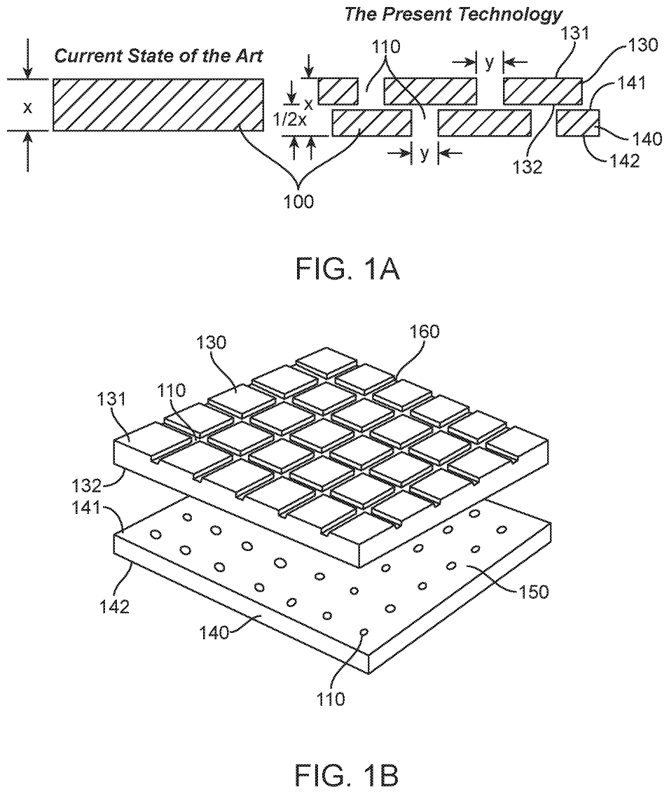

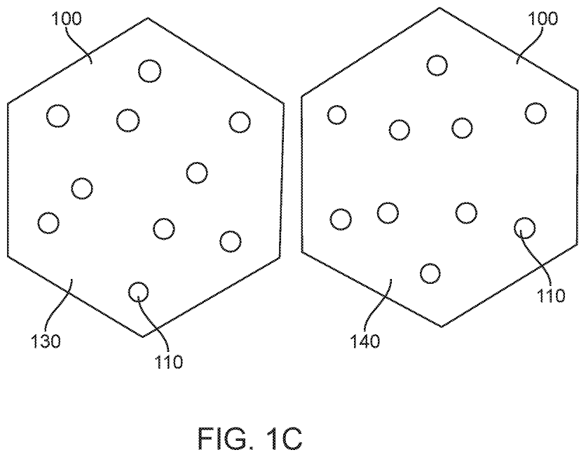

FIGS. 1A-1C provide representative, non-limiting illustrations of embodiments of a wound dressing of the present technology. In FIG. 1A, the difference between the wound dressings of the present technology and the current state of the art is illustrated. Wound dressings currently used in NPWT consist of a single sheet of a biopolymer 100. The wound dressings of the present technology, illustrated in FIG. 1A, comprise multiple layers of biopolymer 100, including a first layer 130 and a second layer 140, and a plurality of perforations 110. Each perforation may include a width (y) and an external edge. The first layer 130 includes a wound-facing side 132 and an environmental-facing side 131. The second layer 140 independently includes a wound-facing side 142 and an environmental-facing side 141. The first layer 130 and the second layer 140 of biopolymer 100 are mated to each other such that the external edge of each perforation 110 in each of the layers overlap or intersect up to about 50%, and that the wound-facing side of the first layer 132 is mated to the environmental-facing side of the second layer 141. Turning to FIG. 1B, the first layer 130 may further include channels 160, wherein each channel may be configured to intersect with at least one perforation 110 along the environmental-facing side of the first layer 131. Further, the first layer 130 and the second layer 140 may be adjoined with a solution 150, that may include water, ethanol, acetic acid, a sugar, a liquid alginate solution, or any combination thereof.

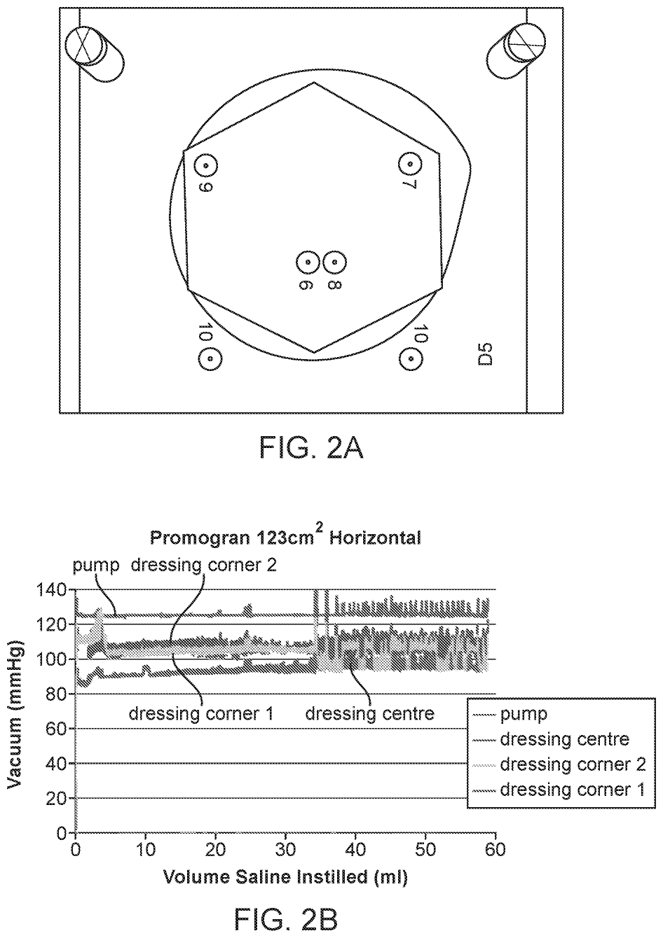

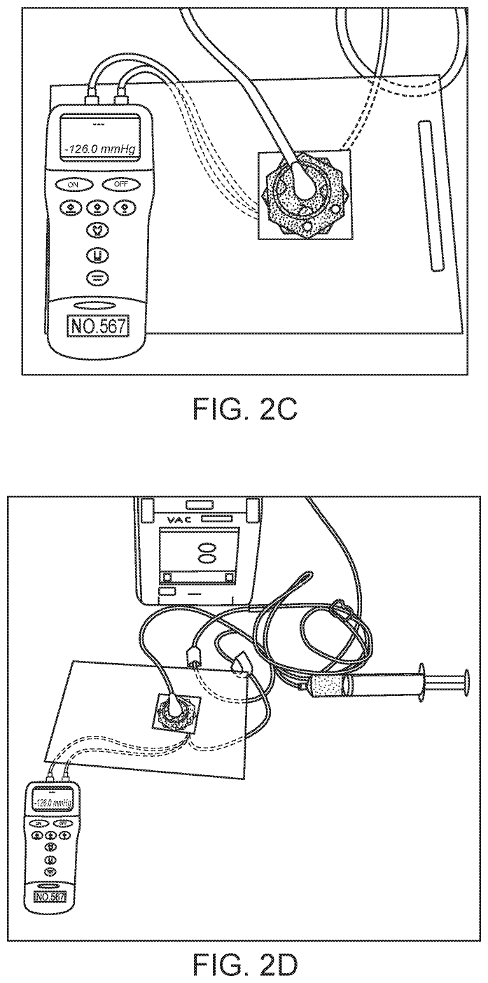

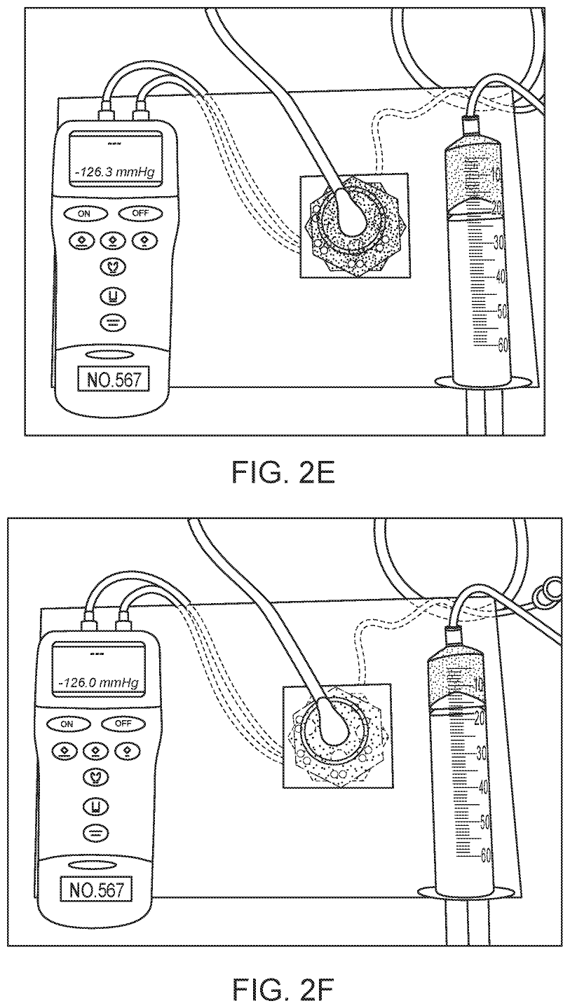

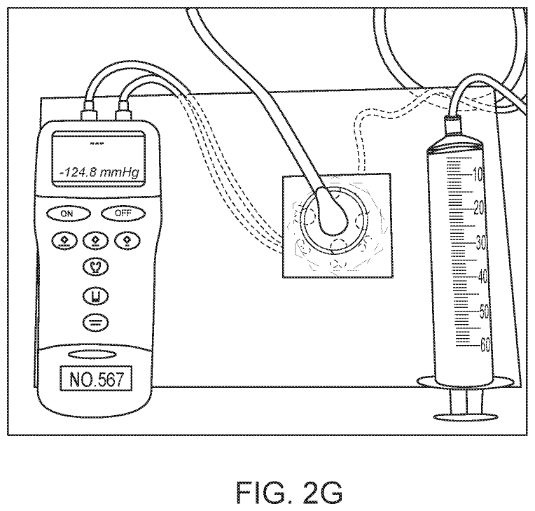

In FIGS. 2A-2B, a control, non-perforated biopolymer 100 (collagen/ORC) wound dressing underwent testing to determine to vacuum pressure at various points across the wound dressing, including the center of the dressing, two different corners of the dressing, and the vacuum pump itself (FIG. 2A). FIG. 2B illustrates that under negative pressure, inconsistent vacuum pressure is observed at various points across the control biopolymer 100 wound dressing. In FIG. 2C, the experimental setup is demonstrated with the perforated, multilayer biopolymer 100 wound dressing of the present technology. In this system, the wound dressing was placed on a layer of polyurethane foam to allow negative pressure to manifold prior to measurement. The V.A.C.® Therapy system was set to 125 mm Hg, with no saline instilled into the setup, and the pressure recorded was 126 mm Hg (FIGS. 2C-2D). Following the delivery of 10 mL of saline (FIG. 2E) to the wound dressing, the perforations 110 increased in translucency which increased the manifolding capabilities of the biopolymer 100, and the pressure applied to the system stabilized to 126.3 mm Hg. An additional 5 mL of saline was then added to the system (FIG. 2F), and following a 10-minute swell period, the pressure applied across the wound dressing of the present technology stabilized to 126 mm Hg. The pressure was then monitored for 3 h after the delivery of the saline (30 mL total), and the pressure stabilized to 124.8 mm Hg, with no detected reduction in pressure (FIG. 2G).

### Definitions

The definitions of certain terms as used in this specification are provided below. Unless defined otherwise, all technical and scientific terms used herein generally have the same meaning as commonly understood by one of ordinary skill in the art to which this present technology belongs.

The following terms are used throughout as defined below.

As used herein and in the appended claims, singular articles such as “a”, “an”, and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, the “administration” of a wound dressing to a subject includes any route of introducing or delivering to a subject a wound dressing to perform its intended function. Administration can be carried out by any suitable route, including but not limited to, topical administration. Administration includes self-administration and the administration by another.

As used herein, the terms “contain”, “contains”, or “containing” in the context of describing the elements (especially in the context of the following claims) are to be construed as comprising or including the elements being described herein.

As used herein, the term “effective amount” refers to a quantity sufficient to achieve a desired therapeutic effect, e.g., an amount which results in wound healing or a reduction of one or more signs or symptoms associated with a wound described herein. In the context of therapeutic applications, the wound dressing administered to the subject will vary depending on the composition, the degree, type, and severity of the wound and on the characteristics of the individual.

As used herein, the terms “individual”, “patient”, or “subject” can be an individual organism, a vertebrate, a mammal, or a human. In some embodiments, the individual, patient or subject is a human.

As understood by one of ordinary skill in the art, “molecular weight” (also known as “relative molar mass”) is a dimensionless quantity that can be converted to molar mass by multiplying by 1 gram/mole—for example, collagen with a weight-average molecular weight of 5,000 has a weight-average molar mass of 5,000 g/mol.

As used herein, the term “NPWT” refers to negative pressure wound therapy, which is a type of wound therapy that involves applying negative pressure (relative to atmospheric pressure) to a wound bed to promote wound healing. Typically, a dressing is sealed over a wound site and air is pumped out of the dressing to create negative pressure at the wound site. In some NPWT systems, wound exudate and other fluid is pumped out of the dressing and collected by a canister.

As used herein, the term “manifold” or “manifolding” generally includes any composition or structure providing a plurality of pathways and/or perforations configured to collect or distribute fluid and/or pressure across a tissue site while under pressure.

As used herein, the term “pitch” refers to the distance between repeated elements in a structure. For example, a pitch between perforations is the distance between one perforation and another perforation within the same layer of the wound dressing. As used herein, the term “inter-layer pitch” refers to the distance between one perforation and another perforation within different layers of the wound dressing.

As used herein, the term “solid content” refers to the density of a material and/or film of the wound dressing or reduced-pressure wound dressing apparatus of the present technology, which is its mass per unit volume.

“Treating” or “treatment” as used herein covers the treatment of a wound described herein, in a subject, such as a human, and includes: (i) inhibiting a wound, i.e., arresting its development; (ii) relieving a wound, i.e., causing regression of the wound; (iii) slowing progression of the wound; and/or (iv) inhibiting, relieving, or slowing progression of one or more symptoms of the wound. In some embodiments, treatment means that the symptoms associated with the wound are, e.g., alleviated, reduced, cured, or placed in a state of remission.

As used herein, the term “% w/v” refers to the percent of weight of the solute in the total volume of the solution, i.e., the number of grams of solute in 100 mL of solution.

It is also to be appreciated that the various modes of treatment of wounds as described herein are intended to mean “substantial,” which includes total but also less than total treatment, and wherein some biologically or medically relevant result is achieved. The treatment may be a continuous prolonged treatment for a chronic wound or a single, or several administrations for the treatment of an acute wound.

### The Wound Dressing of the Present Technology

### The First Layer

The present disclosure provides a wound dressing comprising a first layer 130 and a second layer 140, wherein the first layer 130 may comprise a biopolymer 100 and a plurality of perforations 110.

In any embodiment of the wound dressing disclosed herein, the first layer 130 may include a wound-facing side 132 and an environmental-facing side 131.

In any embodiment of the wound dressing disclosed herein, the first layer 130 may have a thickness of about 1 mm to about 3 mm. Additionally or alternatively, in some embodiments of the wound dressing disclosed herein, the first layer 130 may have a thickness of about 1 mm, about 1.5 mm, about 2 mm, about 2.5 mm, about 3 mm, or any range including and/or in between any two of the preceding values.

In any embodiment of the wound dressing disclosed herein, the biopolymer 100 of the first layer 130 may be one or more of a collagen, an oxidized cellulose, a polysaccharide, chitosan, gelatin, hyaluronic acid, or any combination thereof.

In any embodiment of the wound dressing disclosed herein, the first layer 130 may comprise about 0.1 wt. % to about 100 wt. % biopolymer 100. Additionally or alternatively, in some embodiments of the wound dressing disclosed herein, the amount of biopolymer 100 in the first layer 130 may be about 0.1 wt. %, about 0.5 wt. %, about 1 wt. %, about 2.5 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 98 wt. %, about 99 wt. %, about 100 wt. %, or any range including and/or in between any two of the preceding values.

In any embodiment of the wound dressing disclosed herein, the first layer 130 may comprise a plurality of perforations 110. Additionally or alternatively, in some embodiments of the wound dressing disclosed herein, each perforation 110 may comprise a width and an external perimeter. Additionally or alternatively, in some embodiments of the wound dressing disclosed herein, the width of each perforation 110 in the first layer 130 may independently be at least 1 mm. Additionally or alternatively, in some embodiments of the wound dressing disclosed herein, the width of each perforation 110 in the first layer 130 may independently be about 2 mm to about 5 mm. Additionally or alternatively, in some embodiments of the wound dressing disclosed herein, the width of each perforation 110 in the first layer 130 may independently be about 2 mm, about 2.5 mm, about 3 mm, about 3.5 mm, about 4 mm, about 4.5 mm, about 5 mm, or any range including and/or in between any two of these values.

In any embodiment of the wound dressing disclosed herein, the perforations 110 in the first layer 130 may have a pitch of about 5 mm to about 10 mm. Additionally or alternatively, in some embodiments of the wound dressing disclosed herein, the perforations 110 in the first layer 130 may have a pitch of about 5 mm, about 5.5 mm, about 6 mm, about 6.5 mm, about 7 mm, about 7.5 mm, about 8 mm, about 8.5 mm, about 9 mm, about 9.5 mm, about 10 mm, or any range including and/or in between any two of these values. Additionally or alternatively, in some embodiments, the perforations 110 in the first layer 130 may have a pitch of about 10 mm.

In any embodiment of the wound dressing disclosed herein, the first layer 130 may comprise about 5% to about 50% perforations 110 by area. Thus, the first layer 130 may comprise about 5%, about 10%, about 15%, about 20%, about 25%, about 30%, about 35%, about 40%, about 45%, about 50% by area, or any range including and/or in between any two of these values, perforations 110.

In any embodiment of the wound dressing disclosed herein, the shape of the perforations 110 in the first layer 130 may independently be a circle, a triangle, a quadrilateral, or a polygon (e.g., a pentagon, a hexagon, a heptagon, an octagon, a nonagon, or a decagon).

In any embodiment of the wound dressing disclosed herein, the first layer 130 may comprise a plurality of channels 160, wherein each channel 160 is configured to intersect with at least one perforation 110 along the environmental-facing side 131 of the first layer 130. In any embodiment of the wound dressing disclosed herein, the channels 160 may be compressed to a depth of about 5% to about 50% of the thickness of the first layer 130. Additionally or alternatively, in some embodiments of the wound dressing disclosed herein, the channels 160 may be compressed to a depth of about 5%, about 10%, about 15%, about 20%, about 25%, about 30%, about 35%, about 40%, about 45%, about 50%, or any range including and/or in between any two of these values, relative to the thickness of the first layer 130.

In any embodiment of the wound dressing disclosed herein, the width of the channels 160 may be about 1 mm to about 3 mm. Additionally or alternatively, in some embodiments of the wound dressing disclosed herein, the width of the channels 160 may be about 1 mm, about 1.25 mm, about 1.5 mm, about 1.75 mm, about 2 mm, about 2.25 mm, about 2.5 mm, about 2.75 mm, about 3 mm, or any range including and/or in between any two of the preceding values.

In any embodiment of the wound dressing disclosed herein, the first layer 130 may include channels 160 on about 5% to about 30% of the surface of the first layer 130. Additionally or alternatively, in some embodiments of the wound dressing disclosed herein, the first layer 130 may channels 160 on about 5%, about 10%, about 15%, about 20%, about 25%, about 30%, or any range including and/or in between any two of these values, of the surface of the first layer 130.

In any embodiment of the wound dressing disclosed herein, the solid content of the first layer 130 may comprise about 0.1 wt. % to about 10 wt. %. Additionally or alternatively, in some embodiments of the wound dressing disclosed herein, the solid content of the first layer 130 may comprise about 0.1 wt. %, about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, or any range including and/or in between any two of the preceding values.

In any embodiment of the wound dressing disclosed herein, the first layer 130 may comprise a silver compound. Additionally or alternatively, in some embodiments of the wound dressing disclosed herein, the first layer 130 may comprise about 0.1 wt. % to about 3 wt. % of a silver compound. Additionally or alternatively, in some embodiments of the wound dressing disclosed herein, the first layer 130 may comprise about 0.1 wt. %, about 0.25 wt. %, about 0.50 wt. %, about 0.75 wt. %, about 0.8 wt. %, about 0.85 wt. %, about 0.9 wt. %, about 0.95 wt. %, about 1 wt. %, about 1.25 wt. %, about 1.5 wt. %, about 1.75 wt. %, about 2 wt. %, about 2.25 wt. %, about 2.5 wt. %, about 2.75 wt. %, about 3 wt. %, or any range including and/or in between any two of the preceding values, of the silver compound.

In any embodiment of the wound dressing disclosed herein, the silver compound of the first layer 130 comprises one or more pharmaceutically acceptable silver salts. Exemplary sources of the one or more pharmaceutically acceptable silver salts of the first layer 130 include, but are not limited to, silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver p-aminobenzoate, silver p-aminosalicylate, nanocrystalline silver, any pharmaceutically acceptable salt thereof, or any combination thereof. Additionally or alternatively, in some embodiments of the wound dressing disclosed herein, the silver compound of the first layer 130 comprises a silver (II) oxide, silver (III) oxide, a silver oxy-salt, or any combination thereof. Additionally or alternatively, in some embodiments of the wound dressing disclosed herein, the silver oxy-salt may comprise a general formula of Ag(Ag₃Oₐ)X, wherein X can include, but is not limited to, one or more acid anions such as sulfates, chlorides, phosphates, carbonates, citrates, tartrates, or oxalates; and wherein a is at least two.

In any embodiment of the wound dressing disclosed herein, the first layer 130 may comprise about 1 wt. % to about 15 wt. % of at least one plasticizer. Additionally or alternatively, in some embodiments of the wound dressing disclosed herein, the first layer 130 may comprise about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, about 15 wt. %, or any range including and/or in between any two of the preceding values, of the at least one plasticizer. Exemplary plasticizers include, but are not limited to, an acetylated monoglyceride, an alkyl citrate, methyl ricinoleate, glycerol, polyvinylpyrrolidone, and any combination thereof. Examples of alkyl citrates include, but are not limited to, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, and any combination thereof.

### The Second Layer

The present disclosure provides a wound dressing comprising a first layer 130 and a second layer 140, wherein the second layer 140 may comprise a biopolymer 100 and a plurality of perforations 110.

In any embodiment of the wound dressing disclosed herein, the second layer 140 may include a wound-facing side 142 and an environmental-facing side 141.

In any embodiment of the wound dressing disclosed herein, the second layer 140 may have a thickness of about 1 mm to about 3 mm. Additionally or alternatively, in some embodiments of the wound dressing disclosed herein, the second layer 140 may have a thickness of about 1 mm, about 1.5 mm, about 2 mm, about 2.5 mm, about 3 mm, or any range including and/or in between any two of the preceding values.

In any embodiment of the wound dressing disclosed herein, the biopolymer 100 of the second layer 140 may be a collagen, an oxidized cellulose, a polysaccharide, chitosan, gelatin, hyaluronic acid, or any combination thereof.

In any embodiment of the wound dressing disclosed herein, the second layer 140 may comprise about 0.1 wt. % to about 100 wt. % biopolymer 100. Additionally or alternatively, in some embodiments of the wound dressing disclosed herein, the amount of biopolymer 100 in the second layer 140 may be about 0.1 wt. %, about 0.5 wt. %, about 1 wt. %, about 2.5 wt. %, about 5 wt. %, about 10 wt. %, about 15 wt. %, about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, about 75 wt. %, about 80 wt. %, about 85 wt. %, about 90 wt. %, about 95 wt. %, about 98 wt. %, about 99 wt. %, about 100 wt. %, or any range including and/or in between any two of the preceding values.

In any embodiment of the wound dressing disclosed herein, the second layer 140 may comprise a plurality of perforations 110. Additionally or alternatively, in some embodiments of the wound dressing disclosed herein, each perforation 110 may comprise a width and an external perimeter. Additionally or alternatively, in some embodiments of the wound dressing disclosed herein, the width of each perforation 110 in the second layer 140 may independently be at least 1 mm. Additionally or alternatively, in some embodiments of the wound dressing disclosed herein, the width of each perforation 110 in the second layer 140 may independently be about 2 mm to about 5 mm. Additionally or alternatively, in some embodiments of the wound dressing disclosed herein, the width of each perforation 110 in the second layer 140 may independently be about 2 mm, about 2.5 mm, about 3 mm, about 3.5 mm, about 4 mm, about 4.5 mm, about 5 mm, or any range including and/or in between any two of these values.

In any embodiment of the wound dressing disclosed herein, the perforations 110 in the second layer 140 may have a pitch of about 5 mm to about 10 mm. Additionally or alternatively, in some embodiments of the wound dressing disclosed herein, the perforations 110 in the second layer 140 may have a pitch of about 5 mm, about 5.5 mm, about 6 mm, about 6.5 mm, about 7 mm, about 7.5 mm, about 8 mm, about 8.5 mm, about 9 mm, about 9.5 mm, about 10 mm, or any range including and/or in between any two of these values. Additionally or alternatively, in some embodiments, the perforations 110 in the second layer 140 may have a pitch of about 10 mm.

In any embodiment of the wound dressing disclosed herein, the second layer 140 may comprise about 5% to about 50% perforations 110 by area. Thus, the second layer 140 may comprise about 5%, about 10%, about 15%, about 20%, about 25%, about 30%, about 35%, about 40%, about 45%, about 50% by area, or any range including and/or in between any two of these values, perforations 110.

In any embodiment of the wound dressing disclosed herein, the shape of the perforations 110 in the second layer 140 may independently be a circle, a triangle, a quadrilateral, or a polygon (e.g., a pentagon, a hexagon, a heptagon, an octagon, a nonagon, or a decagon).

In any embodiment of the wound dressing disclosed herein, the solid content of the second layer 140 may comprise about 0.1 wt. % to about 10 wt. %. Additionally or alternatively, in some embodiments of the wound dressing disclosed herein, the solid content of the second layer 140 may comprise about 0.1 wt. %, about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, or any range including and/or in between any two of the preceding values.

In any embodiment of the wound dressing disclosed herein, the second layer 140 may comprise a silver compound. Additionally or alternatively, in some embodiments of the wound dressing disclosed herein, the second layer 140 may comprise about 0.1 wt. % to about 3 wt. % of a silver compound. Additionally or alternatively, in some embodiments of the wound dressing disclosed herein, the second layer 140 may comprise about 0.1 wt. %, about 0.25 wt. %, about 0.50 wt. %, about 0.75 wt. %, about 0.8 wt. %, about 0.85 wt. %, about 0.9 wt. %, about 0.95 wt. %, about 1 wt. %, about 1.25 wt. %, about 1.5 wt. %, about 1.75 wt. %, about 2 wt. %, about 2.25 wt. %, about 2.5 wt. %, about 2.75 wt. %, about 3 wt. %, or any range including and/or in between any two of the preceding values, of the silver compound.

In any embodiment of the wound dressing disclosed herein, the silver compound of the second layer 140 comprises one or more pharmaceutically acceptable silver salts. Exemplary sources of the one or more pharmaceutically acceptable silver salts of the second layer 140 include, but are not limited to, silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver p-aminobenzoate, silver p-aminosalicylate, nanocrystalline silver, any pharmaceutically acceptable salt thereof, or any combination thereof. Additionally or alternatively, in some embodiments of the wound dressing disclosed herein, the silver compound of the second layer 140 comprises a silver (II) oxide, silver (III) oxide, a silver oxy-salt, or any combination thereof. Additionally or alternatively, in some embodiments of the wound dressing disclosed herein, the silver oxy-salt may comprise a general formula of Ag(Ag₃Oₐ)X, wherein X can include, but is not limited to, one or more acid anions such as sulfates, chlorides, phosphates, carbonates, citrates, tartrates, or oxalates; and wherein a is at least two.

In any embodiment of the wound dressing disclosed herein, the second layer 140 may comprise about 1 wt. % to about 15 wt. % of at least one plasticizer. Additionally or alternatively, in some embodiments of the wound dressing disclosed herein, the second layer 140 may comprise about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, about 15 wt. %, or any range including and/or in between any two of the preceding values, of the at least one plasticizer. Exemplary plasticizers include, but are not limited to, an acetylated monoglyceride, an alkyl citrate, methyl ricinoleate, glycerol, polyvinylpyrrolidone, and any combination thereof. Examples of alkyl citrates include, but are not limited to, triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, and any combination thereof.

### The Wound Dressing

In any embodiment of the wound dressing disclosed herein, the wound-facing side 132 of the first layer 130 may be coupled with the environmental-facing side 141 of the second layer 140.

In any embodiment of the wound dressing disclosed herein, wherein the perforations 110 in each of the first layer 130 and the second layer 140 may have a pitch of at least about 10 mm, the wound dressing may have an inter-layer pitch of at least about 5 mm.

In any embodiment of the wound dressing disclosed herein, the first layer 130 and the second layer 140 are may be adjoined with a solution 150. Additionally or alternatively, in some embodiments of the wound dressing disclosed herein, the solution 150 may be water, ethanol, acetic acid, a sugar, a liquid alginate solution, or any combination thereof. Additionally or alternatively, in some embodiments of the wound dressing disclosed herein, the solution 150 may be applied to the wound dressing via spray application or screen printing. Additionally or alternatively, in some embodiments of the wound dressing disclosed herein, the wound dressing may be freeze-dried re-freeze dried after the solution 150 is applied to adjoin the first layer 130 and the second layer 140.

In some embodiments of the wound dressing disclosed herein, the first layer 130 and the second layer 140 maybe coupled such that the external perimeter of each perforation in the each of the first layer 130 and the second layer 140 do not overlap, or overlap or intersect up to about 50%. Thus, the first layer 130 and the second layer 140 maybe coupled such that the external perimeter of each perforation in the each of the first layer 130 and the second layer 140 overlap or intersect 0%, about 5%, about 10%,. about 15%, about 20%, about 25%, about 30%, about 35%, about 40%, about 45%, about 50%, or any range including and/or in between any two of the preceding values.

In any embodiment of the wound dressing disclosed herein, the solution 150 may include at least one additive. Exemplary sources of the at least one additive include, but are not limited to, antimicrobial agents, antioxidants, signaling proteins, or any combination thereof.

In any embodiment of the wound dressing disclosed herein, the solution 150 may comprise an antimicrobial agent. Additionally or alternatively, in some embodiments of the wound dressing disclosed herein, the solution 150 may comprise about 0.01% w/v to about 9% w/v of the antimicrobial agent. Additionally or alternatively, in some embodiments of the wound dressing disclosed herein, the antimicrobial agent may comprise about 0.01% w/v, about 0.1% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, or any range including and/or in between any two of these values. Additionally or alternatively, in some embodiments of the wound dressing disclosed herein, the antimicrobial agent may be one or more of tetracycline, penicillins, terramycins, erythromycin, bacitracin, neomycin, polymycin B, mupirocin, clindamycin, colloidal silver, silver sulfadiazine, chlorhexidine, povidone iodine, triclosan, sucralfate, quaternary ammonium salts, pharmaceutically acceptable silver salts, or any combination thereof.

In any embodiment of the wound dressing disclosed herein, the solution 150 may comprise an antioxidant. Additionally or alternatively, in some embodiments of the wound dressing disclosed herein, the solution 150 may comprise about 0.1% w/v to about 9% w/v of the antioxidant. Additionally or alternatively, in some embodiments of the wound dressing disclosed herein, the antioxidant may comprise about 0.1% w/v, about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, or any range including and/or in between any two of these values. Additionally or alternatively, in some embodiments of the wound dressing disclosed herein, the antioxidant may be one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or any combination thereof.

In any embodiment of the wound dressing disclosed herein, the anthocyanins are selected from the group consisting of cyanidin, delphinidin, malvidin, pelargonidin, peonidin, petunidin, and mixtures thereof.

In any embodiment of the wound dressing disclosed herein, the flavanols are selected from the group consisting of catechin, epicatechin, theaflavin, thearubigins, gallocatechin, epigallocatechin, or any gallate ester thereof, and mixtures thereof.

In any embodiment of the wound dressing disclosed herein, the flavanones are selected from the group consisting of eriodictyol, hesperetin, naringenin, and mixtures thereof.

In any embodiment of the wound dressing disclosed herein, the flavones are selected from the group consisting of apigenin, luteolin, tangeritin, and mixtures thereof.

In any embodiment of the wound dressing disclosed herein, the flavonols are selected from the group consisting of isorhamnetin, kaempferol, myricetin, proanthocyanidins, quercetin, rutin, and mixtures thereof.

In any embodiment of the wound dressing disclosed herein, the isoflavone phytoestrogens are selected from the group consisting of daidzein, genistein, glycitein, and any combination thereof.

In any embodiment of the wound dressing disclosed herein, the phenolic acids are selected from the group consisting of chicoric acid, chlorogenic acid, cinnamic acid, ellagic acid, ellagitannins, gallic acid, gallotannins, rosmarinic acid, salicylic acid, or any ester thereof, and any combination thereof.

In any embodiment of the wound dressing disclosed herein, the stillbenoids are selected from the group consisting of resveratrol, pterostilbene, and any combination thereof.

In any embodiment of the wound dressing disclosed herein, the solution 150 may comprise a signaling protein. Additionally or alternatively, in some embodiments of the wound dressing disclosed herein, the solution 150 may comprise about 1% w/v to about 9% w/v of the signaling protein. Additionally or alternatively, in some embodiments of the wound dressing disclosed herein, the signaling protein may comprise about 1% w/v, about 1.5% w/v, about 2% w/v, about 2.5% w/v, about 3% w/v, about 3.5% w/v, about 4% w/v, about 4.5% w/v, about 5% w/v, about 5.5% w/v, about 6% w/v, about 6.5% w/v, about 7% w/v, about 7.5% w/v, about 8% w/v, about 8.5% w/v, about 9% w/v, or any range including and/or in between any two of these values. Additionally or alternatively, in some embodiments of the wound dressing disclosed herein, the signaling protein may be one or more of platelet-derived growth factor (PDGF), transforming growth factor beta (TGFβ), fibroblast growth factors (FGFs), epidermal growth factor (EGF), or any combination thereof. Additionally or alternatively, in some embodiments of the wound dressing disclosed herein, the fibroblast growth factors (FGFs) may be one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGFS), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or any combination thereof.

In any embodiment disclosed herein, the wound dressing of the present technology may be sterile and packaged in a microorganism-impermeable container.

In any embodiment of the wound dressing disclosed herein, the wound dressing of the present technology is configured for use in negative pressure wound therapy (NPWT). Additionally or alternatively, in some embodiments, NPWT may be performed such as by procedures described in U.S. Pat. Nos. 7,534,240 and 9,918,733, the entire contents of which are incorporated by reference.

In any embodiment of the wound dressing disclosed herein, the application of the wound dressing of the present technology causes about 50% to about 100% reduction in pressure drop observed in negative pressure wound therapy compared to that observed with a control wound dressing that does not comprise a plurality of perforations. Additionally or alternatively, in some embodiments of the wound dressing disclosed herein, the application of the wound dressing of the present technology causes about 50%, about 52%, about 54%, about 56%, about 58%, about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, about 100%, or any range including and/or in between any two of these values, reduction in the pressure drop observed in negative pressure wound therapy compared to that observed with a control wound dressing.

In any embodiment disclosed herein, the wound dressings of the present technology advantageously exhibit improved manifolding and decreased pressure drop observed in NPWT. Without wishing to be bound by theory, it is believed that the wound dressings of the present technology are able to impart constant pressure distribution across a wound site upon application. Further, the wound dressings of the present technology exhibit increased contact across the surface of a wound.

In any embodiment disclosed herein, the wound dressing may be mated to a retainer layer while in use for NPWT. The retainer layer may be configured to be adjoined to the environmental-facing side of the wound dressing of the present technology. The retainer layer may include, but is not limited to, a cellular foam, an open-cell foam, a reticulated foam, porous tissue collections, and/or other porous material (e.g., gauze). The retainer layer may have pores that range in diameter from about 60 μm to about 2000 μm. Thus, the retainer layer may have pores that range in diameter from about 60 μm, about 100 μm, about 250 μm, about 500 μm, about 750 μm, about 1000 μm, about 1250 μm, about 1500 μm, about 1750 μm, about 2000 μm, or any range including and/or in between any two of these values. In some embodiments, the retainer layer may include an open-cell, reticulated polyurethane foam such as a GRANUFOAM™ dressing available from Kinetic Concepts, Inc. of San Antonio, Tex. In some embodiments, the retainer layer may include an open-cell, reticulated polyurethane foam such as a V.A.C. VERAFLO™ dressing available from Kinetic Concepts, Inc. of San Antonio, Tex.

In any embodiment disclosed herein, the drape may be composed of a polyurethane film or an elastomeric film. The drape may be applied over the wound dressing of the present technology and/or the retainer layer during NPWT. The drape may be configured to seal the wound dressing and/or the retainer layer, and the wound site during NPWT. Examples of an elastomeric film include, but are not limited to, natural rubber, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, ethylene vinyl acetate (EVA) film, co-polyester, or silicone. Suitable drape materials and methods of use are described in U.S. Pat. Nos. 7,534,240, 7,611,500, 9,918,733, and U.S. patent application Ser. No. 14/708,078, of which the entire contents are incorporated herein by reference.

In any embodiment disclosed herein, the wound dressing may be connected to tubing while in use for NPWT. The tubing may include, but is not limited to, a tube, pipe, hose, conduit, or any other structure with one or more lumina adapted to convey liquid between two ends. Additionally or alternatively, in some embodiments, the tubing may be composed of polyvinyl chloride, polyethylene, polypropylene, or any combination thereof. The tubing may be configured to connect the drape to a vacuum, such as a V.A.C.® Therapy system, while in use for NPWT. Suitable tubing materials and methods of use are described in U.S. Pat. Nos. 7,534,240, 7,611,500, 9,918,733, and U.S. patent application Ser. No. 14/708,078, of which the entire contents are incorporated herein by reference.

In any embodiment disclosed herein, the wound dressing may be fluidly coupled to a vacuum via the tubing to apply negative pressure to a wound in need thereof. Additionally or alternatively, in some embodiments, negative pressure refers to a pressure less than local ambient pressure, such as the pressure in a local environment external to a sealed wound site. Additionally or alternatively, in some embodiments, the vacuum for applying negative pressure may be a vacuum pump, a suction pump, a micro-pump, or a wall vacuum port available in many healthcare facilities. Additionally or alternatively, in some embodiments, the vacuum is used to apply negative pressure to a wound. Additionally or alternatively, in some embodiments, the negative pressure applied to a wound may be about −5 mm Hg to about −500 mm Hg, or about −75 mm Hg to about −300 mm Hg. Thus, the negative pressure applied to a wound may be about −5 mm Hg, about −25 mm Hg, about −50 mm Hg, about −75 mm Hg, about −100 mm Hg, about −125 mm Hg, about −150 mm Hg, about −175 mm Hg, about −200 mm Hg, about −225 mm Hg, about −250 mm Hg, about −275 mm Hg, about −300 mm Hg, about −325 mm Hg, about −350 mm Hg, about −375 mm Hg, about −400 mm Hg, about −425 mm Hg, about −450 mm Hg, about −475 mm Hg, about −500 mm Hg, or any range including and/or in between any two of these values. Methods of use of negative pressure therapy devices are described in U.S. Pat. Nos. 7,534,240, 7,611,500, 9,918,733, and U.S. patent application Ser. No. 14/708,078, of which the entire contents are incorporated herein by reference.

### Therapeutic Methods of the Present Technology

In one aspect, the present disclosure provides a method for treating a wound in a subject in need thereof, wherein the method comprises administering to the wound a wound dressing of any embodiment disclosed herein. Additionally or alternatively, in some embodiments of the methods disclosed herein, the wound may be an acute wound or a chronic wound. Additionally or alternatively, in some embodiments of the methods disclosed herein, the wound is an acute wound selected from the group consisting of burns, skin grafts, and dehisced surgical wounds. Additionally or alternatively, in some embodiments of the methods disclosed herein, the wound is a chronic wound selected from the group consisting of infectious wounds, venous ulcers, arterial ulcers, decubitis ulcers and diabetic ulcers.

In another aspect, the present disclosure provides a method for treating a wound in a subject in need thereof, wherein the method includes providing a device to the wound, wherein the device includes a wound dressing of any embodiment disclosed herein, optionally a retainer layer, a drape, and a vacuum for applying negative pressure to the wound, wherein the vacuum is configured to be fluidly connected to the drape through tubing; administering to the wound the wound dressing, applying the retainer layer over the wound dressing, and applying the drape over the wound dressing and/or the retainer layer, wherein the drape is configured to seal the wound dressing and/or the retainer layer and the wound site.

Any method known to those in the art for administering a wound dressing to an acute wound or a chronic wound disclosed herein may be employed. Suitable methods include in vitro or in vivo methods. In vivo methods typically include the administration of one or more wound dressings to a subject in need thereof, suitably a human. In some embodiments of the methods disclosed herein, the wound dressing is applied directly to the wound. When used in vivo for therapy, the one or more wound dressings described herein are administered to the subject in effective amounts (i.e., amounts that have desired therapeutic effect). The dose and dosage regimen will depend upon the state of the wound of the subject, and the characteristics of the particular wound dressing used.

The effective amount may be determined during pre-clinical trials and clinical trials by methods familiar to physicians and clinicians. An effective amount of one or more wound dressings useful in the methods may be administered to a subject in need thereof by any number of well-known methods for administering wound dressings.

In some embodiments of the methods disclosed herein, the wound dressings are administered daily for 1 hour or more, for 2 hours or more, for 3 hours or more, for 4 hours or more, for 5 hours or more, for 6 hours or more, for 12 hours or more. Additionally or alternatively, in some embodiments of the methods disclosed herein, the wound dressings are administered one, two, three, four, or five times per day. Additionally or alternatively, in some embodiments of the methods disclosed herein, the wound dressings are administered daily for one, two, three, four or five weeks. Additionally or alternatively, in some embodiments of the methods disclosed herein, the wound dressings are administered daily for less than 6 weeks. Additionally or alternatively, in some embodiments of the methods disclosed herein, the wound dressings are administered daily for 6 weeks or more. Additionally or alternatively, in some embodiments of the methods disclosed herein, the wound dressings are administered daily for 12 weeks or more. Additionally or alternatively, in some embodiments of the methods disclosed herein, the wound dressings are administered every day, every other day, every third day, every fourth day, every fifth day, or every sixth day. Additionally or alternatively, in some embodiments of the methods disclosed herein, the wound dressings are administered weekly, bi-weekly, tri-weekly, or monthly. Additionally or alternatively, in some embodiments of the methods disclosed herein, the wound dressings are administered for a period of one, two, three, four, or five weeks. Additionally or alternatively, in some embodiments of the methods disclosed herein, the wound dressings are administered for six weeks or more. Additionally or alternatively, in some embodiments of the methods disclosed herein, the wound dressings are administered for twelve weeks or more. Additionally or alternatively, in some embodiments of the methods disclosed herein, the wound dressings are administered for a period of less than one year. Additionally or alternatively, in some embodiments of the methods disclosed herein, the wound dressings are administered for a period of more than one year.

In some embodiments of the methods disclosed herein, the wound dressings can be changed for a chronic wound as appropriate. Additionally or alternatively, in some embodiments of the methods disclosed herein, the wound is a chronic wound selected from the group consisting of infectious wounds, venous ulcers, arterial ulcers, decubitis ulcers and diabetic ulcers.

### Methods of Making the Wound Dressings of the Present Technology

Also disclosed herein are methods for making the wound dressings of the present technology. In one aspect, the present disclosure provides a method for making a wound dressing, providing a first layer 130 and a second layer 140, generating a plurality of perforations 110 in each of the first layer 130 and the second layer 140, wherein each perforation 110 comprises a width and an external perimeter, and combining the first layer 130 and the second layer 140 such that the external perimeter of each perforation in the first layer 130 does not overlap or intersect with the external perimeter of each perforation in the second layer 140. Additionally or alternatively, in some embodiments, the first layer 130 and the second layer 140 may be mated such that the external perimeter of each perforation in the each of the first layer 130 and the second layer 140 overlap or intersect up to about 50% by area.

In some embodiments, for example, a first layer 130 of any embodiment disclosed herein is coated with a light solution 150 of water. Next, a second layer 140 of any embodiment disclosed herein may then be disposed on at least a portion of the first layer 130 which has been coated with the solution. In some embodiments, the plurality of perforations 110 in the first layer 130 do not overlap or intersect with the plurality of perforations 110 in the second layer 140. In some embodiments, the plurality of perforations 110 in the first layer 130 and the second layer 140 overlap or intersect up to about 50% by area. The two layers are then compressed together with light pressure, and the proximity of the two layers, and their subsequent interaction will begin to form a bond between the two layers. The two layers are then dried to produce a wound dressing of the present technology.

### Kits Comprising the Wound Dressings of the Present Technology

In a further related aspect, the present disclosure provides kits that include a wound dressing of any embodiment described herein and instructions for use. The kits of the present technology may also include instructions for treating a wound in a subject in need thereof. The kit may optionally comprise components such as antiseptic wipes, ointment, adhesive tape, tweezers, or scissors.

### EXAMPLES

The present technology is further illustrated by the following Example, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Reduced Pressure Drop During Negative Pressure Wound Therapy with Wound Dressings of the Present Technology

To determine the reduction in pressure drop achieved by the multi-layer, perforated wound dressings of the present technology (55% collagen, 45% ORC) or control composite wound dressings (non-perforated; 55% collagen, 45% ORC) were placed in an INFOV.A.C.™ NPWT system (Kinetic Concepts Inc., San Antonio, Tex.) and the pressure was measured as saline was instilled into the NPWT system. The pressure in the NPWT system was monitored at various points across the control wound dressing as saline was instilled at a rate of 2.5 mL/hour (FIGS. 2A and 2B). The results demonstrate that the control wound dressing experienced a pressure drop of about 25-30 mmHg in the center of the dressing. In contrast, the wound dressing of the present technology significantly reduced the overall pressure drop observed while using the NPWT system in conjunction with the collagen/orc dressing after the instillation of 30 mL of saline (FIGS. 2C-2G).

These results demonstrate that the wound dressings of the present technology exhibit a reduction in pressure drop compared to control collagen/ORC alone. Accordingly, the wound dressings of the present technology are useful for treating acute or chronic wounds in a subject in need thereof, wherein the method comprises administering to the wound a wound dressing of any embodiment disclosed herein.

### Example 2: Reduced Pressure Drop During Negative Pressure Wound Therapy with Wound Dressings of the Present Technology

To determine the reduction in pressure drop achieved by the wound dressings of the present technology or a control wound dressing (containing one or more overlapping and/or intersecting perforations) are placed in an INFOV.A.C.™ NPWT system (Kinetic Concepts Inc., San Antonio, Tex.). The pressure is measured as saline is instilled into the NPWT system. The pressure in the NPWT system is then monitored at various points across the control wound dressing as the saline is instilled at a rate of 2.5 mL/hour (30 mL total saline). It is anticipated that the wound dressings of the present technology exhibit a significantly reduced overall pressure drop while using the NPWT system, and improved wound healing of chronic and/or acute wounds compared to that of the control wound dressing while using NPWT.

Accordingly, the wound dressings of the present technology are useful for treating a wound in a subject in need thereof, wherein the method comprises administering to the wound a wound dressing of any embodiment disclosed herein.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds, compositions, or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third, and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A wound dressing comprising:
a first layer and a second layer;
wherein
each of the first layer and the second layer independently comprise a biopolymer and a plurality of perforations;
each perforation comprises a width and an external perimeter; and
the first layer and the second layer are adjoined such that the external perimeter of each perforation in the first layer does not overlap or intersect with the external perimeter of each perforation in the second layer.

2. The wound dressing of claim 1, wherein the perforations comprise at least 10% of an area independently comprised by each of the first layer and the second layer.

3. The wound dressing of claim 2, wherein each of the first layer and the second layer independently comprise a wound-facing side and an environmental-facing side, wherein the wound-facing side of the first layer is coupled with the environmental-facing side of the second layer.

4. The wound dressing of any one of claims 1-3, wherein the width of each perforation in each of the first layer and the second layer is at least 1 mm.

5. The wound dressing of any one of claims 1-4, wherein the width of each perforation in each of the first layer and the second layer is about 2 mm to about 5 mm.

6. The wound dressing of any one of claims 1-5, wherein the perforations in each of the first layer and the second layer has a pitch of at least about 10 mm.

7. The wound dressing of any one of claims 1-6, wherein the shape of the perforations in each of the first layer and the second layer is independently a circle, a triangle, a quadrilateral, or a polygon (e.g., a pentagon, a hexagon, a heptagon, an octagon, a nonagon, or a decagon).

8. The wound dressing of any one of claims 3-7, wherein the first layer further comprises channels on the environmental-facing side of the first layer, wherein each channel is configured to intersect with at least one perforation along the environmental-facing side of the first layer.

9. The wound dressing of any one of claims 1-8, wherein the biopolymer of each of the first layer and second layer are independently one or more of a collagen, an oxidized cellulose, a polysaccharide, chitosan, gelatin, hyaluronic acid, or any combination thereof.

10. The wound dressing of any one of claims 1-9, wherein each of the first layer and the second layer independently comprise about 0.1 wt. % to about 100 wt. % biopolymer.

11. The wound dressing of any one of claims 1-10, wherein each of the first layer and the second layer independently has a thickness of about 1 mm to about 3 mm.

12. The wound dressing of any one of claims 1-11, wherein one or more of the first layer and the second layer comprises a silver compound.

13. The wound dressing of claim 12, wherein one or more of the first layer and the second layer comprises about 0.1 wt. % to about 3 wt. % of the silver compound.

14. The wound dressing of claim 12 or claim 13, wherein the silver compound comprises one or more pharmaceutically acceptable silver salts.

15. The wound dressing composition of claim 14, wherein the one or more pharmaceutically acceptable silver salts is selected from the group consisting of silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver p-aminobenzoate, silver p-aminosalicylate, nanocrystalline silver, any pharmaceutically acceptable salt thereof, and any combination thereof.

16. The wound dressing of any one of claims 1-15, wherein an adhesive layer is located between the first layer and the second layer.

17. The wound dressing of claim 16, wherein the adhesive layer comprises acetic acid, a sugar, an alginate, or any combination thereof.

18. The wound dressing of claim 16 or claim 17, wherein the adhesive layer is a layer of a plurality of adhesive droplets or is a screen printed adhesive.

19. The wound dressing of any one of claims 16-18, wherein the adhesive layer is a freeze dried adhesive layer.

20. The wound dressing of any one of claims 16-19, wherein the adhesive layer further comprises at least one additive.

21. The wound dressing of claim 20, wherein the at least one additive is an antimicrobial agent, an antioxidant, a signaling protein, or any combination thereof.

22. The wound dressing of claim 21, wherein the adhesive layer further comprises an antimicrobial agent.

23. The wound dressing of claim 22, wherein the adhesive layer comprises about 0.001 wt. % to about 5 wt. % of the antimicrobial agent.

24. The wound dressing of claim 22 or claim 23, wherein the antimicrobial agent comprises one or more of tetracycline, penicillins, terramycins, erythromycin, bacitracin, neomycin, polymycin B, mupirocin, clindamycin, colloidal silver, silver sulfadiazine, chlorhexidine, povidone iodine, triclosan, sucralfate, quaternary ammonium salts, pharmaceutically acceptable silver salts, or any combination thereof.

25. The wound dressing of any one of claims 21-23, wherein the adhesive layer further comprises an antioxidant.

26. The wound dressing of claim 25, wherein the adhesive layer comprises about 0.001 wt. % to about 5 wt. % of the antioxidant.

27. The wound dressing of claim 25 or claim 26, wherein the antioxidant comprises one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or any combination thereof.

28. The wound dressing of claim 27, wherein the anthocyanins are selected from the group consisting of cyanidin, delphinidin, malvidin, pelargonidin, peonidin, petunidin, and any combination thereof.

29. The wound dressing of claim 27, wherein the flavanols are selected from the group consisting of catechin, epicatechin, theaflavin, thearubigins, gallocatechin, epigallocatechin, or any gallate ester thereof, and any combination thereof.

30. The wound dressing of claim 27, wherein the flavanones are selected from the group consisting of eriodictyol, hesperetin, naringenin, and any combination thereof.

31. The wound dressing of claim 27, wherein the flavones are selected from the group consisting of apigenin, luteolin, tangeritin, and any combination thereof.

32. The wound dressing of claim 27, wherein the flavonols are selected from the group consisting of isorhamnetin, kaempferol, myricetin, proanthocyanidins, quercetin, rutin, and any combination thereof.

33. The wound dressing of claim 27, wherein the isoflavone phytoestrogens are selected from the group consisting of daidzein, genistein, glycitein, and any combination thereof.

34. The wound dressing of claim 27, wherein the phenolic acids are selected from the group consisting of chicoric acid, chlorogenic acid, cinnamic acid, ellagic acid, ellagitannins, gallic acid, gallotannins, rosmarinic acid, salicylic acid, or any ester thereof, and any combination thereof.

35. The wound dressing of claim 27, wherein the stilbenoids are selected from the group consisting of resveratrol, pterostilbene, and any combination thereof.

36. The wound dressing of any one of claims 16-35, wherein the solution further comprises a signaling protein.

37. The wound dressing of claim 36, wherein the solution comprises about 0.001 wt. % to about 5 wt. % of the signaling protein.

38. The wound dressing of claim 36 or claim 37, wherein the signaling protein comprises one or more of platelet-derived growth factor (PDGF), transforming growth factor beta (TGFβ), fibroblast growth factors (FGFs), epidermal growth factor (EGF), or any combination thereof.

39. The wound dressing of claim 38, wherein the fibroblast growth factors (FGFs) comprise one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGF5), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or any combination thereof.

40. The wound dressing of any one of claims 1-39, wherein the wound dressing is configured for use in negative pressure wound therapy.

41. The wound dressing of any one of claims 1-40, configured to reduce a pressure drop in negative pressure wound therapy by about 50% to about 100% compared to that observed with:
(a) a control wound dressing that does not comprise a plurality of perforations; or
(b) a control wound dressing comprising a first layer and a second layer, wherein each of the first layer and the second layer independently comprise a plurality of perforations, and wherein at least one perforation in the first layer overlaps or intersects with a perforation in the second layer.

42. A wound dressing comprising:
a negative pressure source;
a wound dressing comprising a first layer and a second layer;
wherein
each of the first layer and the second layer independently comprise a biopolymer and a plurality of perforations;
each perforation comprises a width and an external edge; and
the first layer and the second layer are mated such that the external edge of each perforation in the first layer does not overlap or intersect with the external edge of each perforation in the second layer.

43. An apparatus for treating a wound, wherein the apparatus comprises:
a first layer and a second layer;
wherein
each of the first layer and the second layer independently comprise a biopolymer and a plurality of perforations;
each perforation comprises a width and an external edge; and
the first layer and the second layer are mated such that the external edge of each perforation in the first layer does not overlap or intersect with the external edge of each perforation in the second layer.

44. A method for treating a wound in a subject in need thereof, the method comprising administering to the wound a wound dressing of any one of claims 1-42.

45. The method of claim 44, wherein the wound is an acute wound or a chronic wound.

46. The method of claim 44 or claim 45, wherein the wound dressing is applied directly to the wound.

47. A method for treating a treating a wound in a subject in need thereof, the method comprising administering a negative pressure to the wound and the wound dressing of the apparatus of claim 43.

48. The method of claim 47, wherein the wound is an acute wound or a chronic wound.

49. The method of claim 47 or claim 48, wherein the apparatus is applied directly to the wound.

50. A method for treating a wound in a subject in need thereof, comprising:
providing a device to the wound, wherein the device comprises:
a wound dressing of any one of claims 1-42;
optionally a retainer layer;
a drape; and
a vacuum for applying negative pressure to the wound, wherein the vacuum is configured to be fluidly connected to the drape through tubing,
administering to the wound the wound dressing;
optionally applying the retainer layer over the wound dressing; and
applying the drape over the wound dressing and/or the retainer layer, wherein the drape is configured to seal the wound dressing and/or the retainer layer and the wound site.

51. A method for making a wound dressing:
(a) providing a first layer and a second layer, each layer defining a plurality of perforations in each of the first layer and the second layer, wherein each perforation comprises a width and an external perimeter; and
(b) combining the first layer and the second layer such that the external perimeter of each perforation in the first layer does not overlap or intersect with the external perimeter of each perforation in the second layer.

52. The method of claim 51, wherein the perforations comprise at least 10% of an area independently comprised by each of the first layer and the second layer.

53. The method of claim 52, wherein each of the first layer and the second layer independently comprise a wound-facing side and an environmental-facing side, wherein the wound-facing side of the first layer is coupled with the environmental-facing side of the second layer.

54. The method of any one of claim 51-53, wherein the width of each perforation in each of the first layer and the second layer is at least 1 mm.

55. The method of any one of claims 51-54, wherein the width of each perforation in each of the first layer and the second layer is about 2 mm to about 5 mm.

56. The method of any one of claims 51-55, wherein the perforations in each of the first layer and the second layer has a pitch of at least about 10 mm.

57. The method of any one of claims 51-56, wherein the shape of the perforations in each of the first layer and the second layer is independently a circle, a triangle, a quadrilateral, or a polygon (e.g., a pentagon, a hexagon, a heptagon, an octagon, a nonagon, or a decagon).

58. The method of any one of claims 53-57, wherein the first layer further comprises channels on the environmental-facing side of the first layer, wherein each channel is configured to intersect with at least one perforation along the environmental-facing side of the first layer.

59. The method of any one of claims 51-58, wherein the biopolymer of each of the first layer and second layer are independently a collagen, an oxidized cellulose, a polysaccharide, chitosan, gelatin, hyaluronic acid, or a combination of any two or more thereof.

60. The method of any one of claims 51-59, wherein each of the first layer and the second layer independently comprise about 0.1 wt. % to about 100 wt. % of the biopolymer.

61. The method of any one of claims 51-60, wherein each of the first layer and the second layer independently has a thickness of about 1 mm to about 3 mm.

62. The method of any one of claims 51-61, wherein one or more of the first layer and the second layer comprises a silver compound.

63. The method of claim 62, wherein one or more of the first layer and the second layer comprises about 0.1 wt. % to about 3 wt. % of the silver compound.

64. The method of claim 62 or claim 63, wherein the silver compound comprises one or more pharmaceutically acceptable silver salts.

65. The method of claim 64, herein the one or more pharmaceutically acceptable silver salts is selected from the group consisting of silver oxide, silver chromate, silver allantoinate, silver borate, silver glycerolate, silver nitrate, silver acetate, silver chloride, silver sulfate, silver lactate, silver bromide, silver iodide, silver carbonate, silver citrate, silver laurate, silver deoxycholate, silver salicylate, silver p-aminobenzoate, silver p-aminosalicylate, nanocrystalline silver, any pharmaceutically acceptable salt thereof, and any combination thereof.

66. The method of any one of claims 51-65, wherein the first layer and the second layer are adjoined with a solution.

67. The method of claim 66, wherein the solution comprises water, ethanol, acetic acid, a sugar, a liquid alginate solution, or any combination thereof.

68. The method of claim 66 or claim 67, wherein the solution is applied to the wound dressing via spray application or screen printing.

69. The method of any one of claims 65-67, wherein the wound dressing is freeze dried after the solution is applied, wherein freeze drying adjoins the first layer and the second layer.

70. The wound dressing of any one of claims 66-69, wherein the solution further comprises at least one additive.

71. The wound dressing of claim 70, wherein the at least one additive is an antimicrobial agent, an antioxidant, a signaling protein, or any combination thereof.

72. The method of any one of claims 66-71, wherein the solution further comprises an antimicrobial agent.

73. The method of claim 72, wherein the solution comprises about 0.001 wt. % to about 5 wt. % of the antimicrobial agent.

74. The method of claim 72 or claim 73, wherein the antimicrobial agent comprises one or more of tetracycline, penicillins, terramycins, erythromycin, bacitracin, neomycin, polymycin B, mupirocin, clindamycin, colloidal silver, silver sulfadiazine, chlorhexidine, povidone iodine, triclosan, sucralfate, quaternary ammonium salts, pharmaceutically acceptable silver salts, or any combination thereof.

75. The method of any one of claims 66-74, wherein the solution further comprises an antioxidant.

76. The method of claim 75, wherein the solution comprises about 0.001 wt. % to about 5 wt. % of the antioxidant.

77. The method of claim 75 or claim 76, wherein the antioxidant comprises one or more of anthocyanins, astaxanthin, bilirubin, canthaxanthin, capsaicin, citric acid, curcumin, coenzyme Q10, eugenol, flavanols, flavonolignans, flavanones, flavones, flavonols, iodide, isoflavone phytoestrogens, lutein, lycopene, manganese, melatonin, N-acetylcysteine, oxalic acid, phenolic acids, phytic acid, R-α-lipoic acid, stilbenoids, tocopherol, tocotrienol, vitamin A, vitamin C, vitamin E, xanthones, zeaxanthin, α-carotene, β-carotene, or any combination thereof.

78. The method of claim 77, wherein the anthocyanins are selected from the group consisting of: cyanidin, delphinidin, malvidin, pelargonidin, peonidin, petunidin, and any combination thereof.

79. The method of claim 77, wherein the flavanols are selected from the group consisting of catechin, epicatechin, theaflavin, thearubigins, gallocatechin, epigallocatechin, and any gallate ester thereof, or any combination thereof.

80. The method of claim 77, wherein the flavanones are selected from the group consisting of eriodictyol, hesperetin, naringenin, and any combination thereof.

81. The method of claim 77, wherein the flavones are selected from the group consisting of apigenin, luteolin, tangeritin, and any combination thereof.

82. The method of claim 77, wherein the flavonols are selected from the group consisting of isorhamnetin, kaempferol, myricetin, proanthocyanidins, quercetin, rutin, and any combination thereof.

83. The method of claim 77, wherein the isoflavone phytoestrogens are selected from the group consisting of daidzein, genistein, glycitein, and any combination thereof.

84. The method of claim 77, wherein the phenolic acids are selected from the group consisting of chicoric acid, chlorogenic acid, cinnamic acid, ellagic acid, ellagitannins, gallic acid, gallotannins, rosmarinic acid, salicylic acid, or any ester thereof, and any combination thereof.

85. The method of claim 77, wherein the stilbenoids are selected from the group consisting of resveratrol, pterostilbene, and any combination thereof.

86. The method of any one of claims 66-85, wherein the solution further comprises a signaling protein.

87. The method of claim 86, wherein the solution comprises about 0.001 wt. % to about 5 wt. % of the signaling protein.

88. The method of claim 86 or claim 87, wherein the signaling protein comprises one or more of platelet-derived growth factor (PDGF), transforming growth factor beta (TGFβ), fibroblast growth factors (FGFs), epidermal growth factor (EGF), or any combination thereof.

89. The method of claim 88, wherein the fibroblast growth factors (FGFs) comprise one or more of fibroblast growth factor 1 (FGF1), fibroblast growth factor 2 (FGF2), fibroblast growth factor 3 (FGF3), fibroblast growth factor 4 (FGF4), fibroblast growth factor 5 (FGFS), fibroblast growth factor 6 (FGF6), fibroblast growth factor 7/keratinocyte growth factor (FGF7/KGF), fibroblast growth factor 8 (FGF8), fibroblast growth factor 9 (FGF9), fibroblast growth factor 10/keratinocyte growth factor 2 (FGF10/KGF2), fibroblast growth factor 11 (FGF11), fibroblast growth factor 12 (FGF12), fibroblast growth factor 13 (FGF13), fibroblast growth factor 14 (FGF14), fibroblast growth factor 15 (FGF15), fibroblast growth factor 16 (FGF16), fibroblast growth factor 17 (FGF17), fibroblast growth factor 18 (FGF18), fibroblast growth factor 19 (FGF19), fibroblast growth factor 20 (FGF20), fibroblast growth factor 21 (FGF21), fibroblast growth factor 22 (FGF22), fibroblast growth factor 23 (FGF23), or any combination thereof.

90. The method of any one of claims 51-89, wherein the wound dressing is configured for use in negative pressure wound therapy.

91. A method for making an apparatus for treating a wound, comprising:
(a) providing a first layer and a second layer, each layer defining a plurality of perforations in each of the first layer and the second layer, wherein each perforation comprises a width and an external circumference; and
(b) combining the first layer and the second layer such that the external circumference of each perforation in the first layer does not overlap or intersect with the external circumference of each perforation in the second layer.

92. The method of claim 91, wherein the perforations comprise at least 10% of an area independently comprised by each of the first layer and the second layer.

93. The method of claim 92, wherein each of the first layer and the second layer independently comprise a wound-facing side and an environmental-facing side, the wound-facing side of the first layer adjoined with the environmental-facing side of the second layer.

94. A kit comprising the wound dressing of any one of claims 1-42, and instructions for use.

95. A kit comprising a first solid layer and a second solid layer independently comprising a resorbable biopolymer, each layer independently defining a plurality of perforations, wherein each perforation comprises a width of at least 1 mm and an external circumference.

96. The kit of claim 95, wherein the perforations comprise at least 10% of an area independently comprised by each of the first layer and the second layer.

97. The kit of claim 94 or claim 95, wherein the first layer and second layer are mated together so that the external circumference of each perforation in the first layer does not overlap or intersect with the external circumference of each perforation in the second layer.

98. A kit comprising the apparatus of claim 43, and instructions for use.
